(19) United States
(12) Patent Application Publication
(10) Pub. No.: US 2019/0299005 A1
(43) Pub. Date: Oct. 3, 2019

(54) VAGUS NERVE STIMULATION AND MONITORING

(71) Applicants: John BIENENSTOCK, Toronto (CA); Paul FORSYTHE, Hamilton (CA); Christine WEST, Milton (CA); Wolfgang KUNZE, Hamilton (CA); Karen-Anne NEUFELD, Dundas (CA)

(72) Inventors: John BIENENSTOCK, Toronto (CA); Paul FORSYTHE, Hamilton (CA); Christine WEST, Milton (CA); Wolfgang KUNZE, Hamilton (CA); Karen-Anne NEUFELD, Dundas (CA)

(21) Appl. No.: 16/365,899

(22) Filed: Mar. 27, 2019

(30) Foreign Application Priority Data
Mar. 28, 2018 (SE) 1850351-6

(51) Int. Cl. A61N 1/36

(52) U.S. Cl. CPC A61N 1/36096; A61N 1/36114; A61N 1/36178; A61N 1/36085; A61N 1/36139; A61N 1/36053

(57) ABSTRACT

Vagus nerve stimulation effective in treating mood disorders include application of bursts of vagus nerve stimulation pulses having an average inter-burst duration and an average inter-pulse duration within selected intervals. The effectiveness of test agents in treating mood disorders can be determined by monitoring the firing pattern induced by the test agents on the vagus nerve. Test agents capable of inducing a firing pattern of vagus nerve pulses with average inter-burst duration and average inter-pulse duration within the selected intervals are identified as useful in treating a mood disorder.

### TECHNICAL FIELD

The present embodiments generally relate to vagus nerve stimulation for treating mood disorders, and monitoring vagus nerve activity for screening for test agents.

### BACKGROUND

Several classes of drugs for mood disorders have been developed. The drugs largely rely on the conceptual framework that increases in availability at central nervous system (CNS) synapses of either serotonin (5HT) or noradrenaline would improve mood and/or reverse predicted decreases in 5HT and noradrenaline in patients with these diagnoses. Consequently, selective serotonin reuptake inhibitors (SSRIs) have been developed. It is assumed that the effectiveness of SSRIs in various forms of mood disorders is dependent on their ability to achieve this end. None of the SSRIs are, however, entirely selective in their actions, which vary from inhibition of selective transport of 5HT into cells to prevention of degradation of 5HT. In addition, they have different capacities to increase local levels of 5HT, noradrenaline or dopamine, but the invariable characteristic of SSRIs is that they predominantly affect 5HT levels relative to the other neurotransmitters.

It has always been assumed that if a drug improves mood and psychiatric disorders in general, they must be working directly in the brain. One of the most used SSRIs, fluoxetine (PROZAC® and SARAFEM®) was developed on the basis that it blocked the uptake of 5HT and therefore left more 5HT around for action in the brain.

US 2013/0245486 discloses a device for treating migraine headache by electrically stimulating a vagus nerve situated within a patient's neck.

WO 2007/115113 discloses an apparatus for treating various medical conditions of a patient by detecting a cardiac cycle of the patient and applying an electrical signal to the patient's vagus nerve through an electrode at a selected point in the cardiac or respiratory cycle. The selected point is a point in the cardiac cycle correlated with increased afferent conduction on the vagus nerve or a point in the cardiac cycle when application of the electrical signal increases heart rate variability.

US 2017/0340881 discloses a system of generating and applying a synthetic neuromodulatory signal. Neurogram signals are recorded from a patient put under a particular condition that causes an effect in the patient. The neurogram is then used to create a synthetic neuromodulatory signal that can be applied to a user so that the user experience the same effect as the patient that had been placed in the condition.

US 2008/0269834 discloses an apparatus for providing trans-esophageal electrical signal therapy to a portion of a vagus nerve of a patient to treat a medical condition.

Perez-Burgos et al, American Journal of Physiology-Gastrointestinal and Liver Physiology 304: G211-G220, 2013 discloses that the psychoactive bacteria Lactobacillus rhamnosus (JB-1) elicts rapid frequency facilitation in vagal afferents.

### SUMMARY

It is a general objective to provide a vagus nerve stimulation or firing pattern that mimics the effects obtained by anti-mood disorder treatment.

This and other objectives are met by embodiments as disclosed herein.

The present invention is defined in the independent claims. Further embodiments of the present invention are defined in the dependent claims.

The present invention is based on the finding that certain anti-mood disorder medicaments induce a specific firing pattern of the vagus nerve, i.e., a specific vagus nerve code. The therapeutic effect of these medicaments disappear when the test animals are vagotomized. Application of vagus nerve stimulation pulses according to the vagus nerve code will induce an anti-mood disorder effect in subjects mimicking the anti-mood disorder effect obtained by administration of anti-mood disorder medicaments. Furthermore, the effectiveness of test agents in treating mood disorders can be determined by monitoring the firing pattern induced by the test agents on the vagus nerve. Test agents capable of inducing a firing pattern corresponding to the vagus nerve code of the invention are identified as useful in treating a mood disorder.

The vagus nerve code of the present invention more closely mimics natural afferent vagal signaling, thereby allowing for superior control of mood altering effects evoked by vagus nerve stimulation. The vagus nerve code of the present invention also produces fewer side-effects as compared to prior art vagal stimulation patterns.

### BRIEF DESCRIPTION OF THE DRAWINGS

The embodiments, together with further objects and advantages thereof, may best be understood by making reference to the following description taken together with the accompanying drawings, in which:

FIG. 1 is a schematic block diagram of a device for vagus nerve stimulation according to an embodiment;

FIG. 2 is a schematic block diagram of a device for vagus nerve stimulation according to another embodiment;

FIG. 3 schematically illustrates bursts of vagus nerve stimulation pulses;

FIG. 4 is a flow chart illustrating a method for treating depression according to an embodiment;

FIG. 5 is a flow chart illustrating an ex vivo screening method according to an embodiment;

FIG. 6 is a schematic block diagram of an ex vivo screening system according to an embodiment;

FIG. 7 is a flow chart illustrating an in vivo screening method according to an embodiment;

FIG. 8 is a schematic block diagram of an in vivo screening system according to an embodiment;

FIG. 9 is a flow chart illustrating a method of classifying patients according to an embodiment;

FIG. 10 is a schematic block diagram of a device for classifying patients according to an embodiment;

FIGS. 11A and 11B are diagrams illustrating vagus code parameters following treatment with various antidepressants;

FIGS. 12A and 1B are diagrams comparing vagus code parameters for vagal dependent and vagal independent antidepressants;

FIGS. 13A-13C are diagrams illustrating average inter-pulse durations for various antidepressants;

FIG. 14 is a diagram illustrating Tail Suspension Test (TST) results for various antidepressants in mice;

FIG. 15 is a diagram illustrating TST results for JB-1 antidepressant in mice; and

FIG. 16 is a diagram illustrating average inter-pulse durations for mice orally treated with various antidepressants.

### DETAILED DESCRIPTION

Throughout the drawings, the same reference numbers are used for similar or corresponding elements.

The present embodiments generally relate to vagus nerve stimulation for treating mood disorders, and monitoring vagus nerve activity for screening for test agents.

It is known that most 5HT in the body resides in the gut whether the majority is synthesized from dietary ingestion of the precursor tryptophan. However, it has up until now not been disclosed that the effect of an SSRI might be primarily in the gut itself with subsequent actions in the brain. The present invention is based on the unexpected finding that the SSRI fluoxetine decreased depressive and anxiety-like behavior in a mouse strain known for its anxiety-like behavior, but that the fluoxetine treatment had no effect in mice having been subjected to sub-diaphragmatic vagotomy prior to oral administration of the SSRI fluoxetine. The same dependency of action of other SSRIs, including sertraline (ZOLOFT®), and the bacterial strain Lactobacillus rhamnosus JB-1, known to decrease depressive and anxiety behavior in mice, on presence of vagus nerve was also confirmed. Thus, antidepressant agents were capable of decreasing depressive and anxiety behavior in mice and this effect of the antidepressant agents disappeared when the animals were vagotomized. These findings thereby indicate that SSRI drugs can exert their effect through vagus nerve signaling. By analyzing vagus nerve signals induced by SSRI drugs, a unique vagal firing pattern, denoted vagus nerve code herein, was produced. This vagus nerve code correlates with antidepressant signals.

Thus, processing and decoding of the electrical pulse patterns evoked in the vagus nerve when subjected to antidepressant agents revealed a specific pulse pattern, i.e., the vagus nerve code, which signaled the antidepressant effect.

Using post-hoc analysis of hundreds of action potential recordings, multiple variables or parameters were identified that defined the electrical pulse pattern on the vagus nerve induced by the antidepressant agents. Multivariate analysis of the results showed at least two of the variables or parameters encoded for the observed behavioral effects on anxiety and depression.

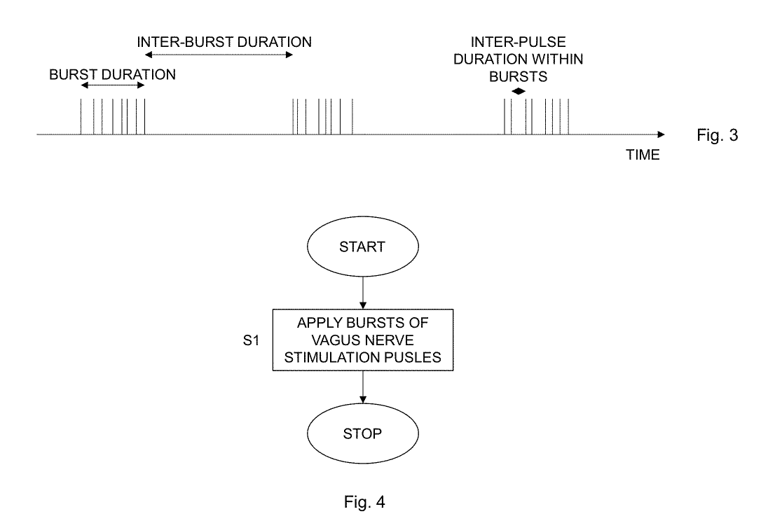

As is schematically illustrated in FIG. 3, the vagus nerve code or pulse pattern consists of bursts of vagus nerve pulses with intermediate more or less silent periods. The vagus nerve pulses could be in the form of applied stimulation pulses, such as when treating a subject suffering from a mood disorder. The vagus nerve pulses could also be bursts of vagus nerve pulses or action potentials in the case of spontaneous firing of the vagus nerve.

In either case, the bursts have an average burst duration as indicated in the figure corresponding to the period in time from the first vagus nerve (stimulation) pulse in a burst up to the last vagus nerve (stimulation) pulse in the same burst. The vagus nerve code or pulse pattern is also characterized by an inter-burst duration, also referred to as inter-burst interval, corresponding to the period in time from the last vagus nerve (stimulation) pulse in a burst up to the first vagus nerve (stimulation) pulse in a subsequent burst. Another parameter or variable is the inter-pulse duration within bursts, also referred to as inter-pulse interval within bursts or intra-burst interval. This parameter or variable corresponds to the period of time between successive vagus nerve (stimulation) pulses within a burst. A further parameter or variable of the vagus nerve code is the inter-pulse duration, also referred to as mean or average inter-pulse interval. This parameter or variable corresponds to the average period of time between vagus nerve (stimulation) pulses throughout the burst train. Thus, the parameter or variable is obtained by dividing the period of time from the start of the first burst to the end of the last burst divided by the total number of vagus nerve (stimulation) pulses occurring during this period of time, i.e., from the start of the first burst to the end of the last burst.

Accordingly, applying bursts of vagus nerve stimulation pulses according to the invention to a subject in need thereof will produce similar antidepressant and/or antianxiety behavior and effect in the subject as administering antidepressant agents and SSRIs to the subject.

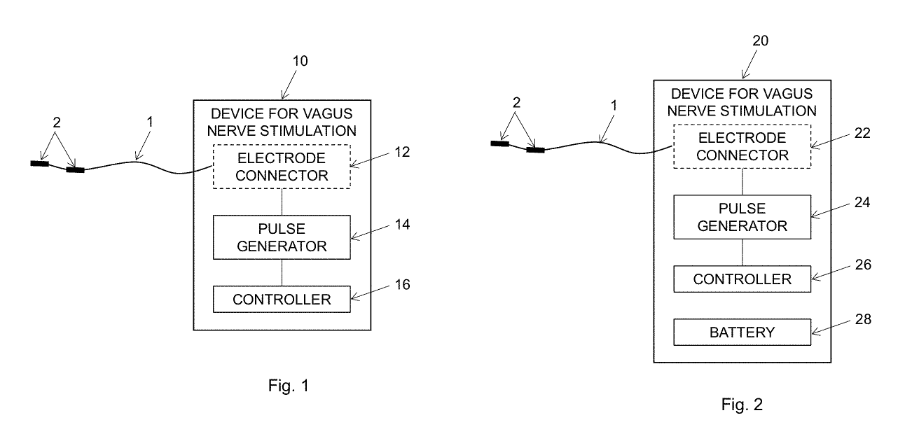

An aspect of the embodiments therefore relates to a device 10, 20 for vagus nerve stimulation, see FIGS. 1 and 2. The device 10, 20 comprises a pulse generator 14, 24 configured to generate vagus nerve stimulation pulses. The device 10, 20 also comprises a controller 16, 26 configured to control the pulse generator 14, 24. In more detail, the controller 16, 26 is configured to control the pulse generator 14, 24 to generate bursts of vagus nerve stimulation pulses having an average inter-burst duration (average inter-burst interval) selected within an interval of from 6 300 to 49 000 ms and an average inter-pulse duration (average inter-pulse interval) selected within an interval of from 180 to 1 600 ms.

This means that the controller 16, 26 controls the pulse generator 14, 24 to generate vagus nerve stimulation pulses according to the vagus nerve code that is, in a general embodiment, defined by the parameters or variables average inter-burst duration and average inter-pulse duration.

The average inter-burst duration should be within an interval of 6 300 and 49 000 ms. In a preferred embodiment, the controller 16, 26 is configured to control the pulse generator 14, 24 to generate the bursts of vagus nerve stimulation pulses having the average inter-burst duration selected within an interval of from 10 000 to 45 000 ms, more preferably within an interval of from 21 500 to 30 100 ms.

Experimental data generated for the three antidepressant agents fluoxetine, sertraline and JB-1 shows that the lower 95% confidence interval (CI) of the average or mean inter-burst duration is 21 582 ms and the upper 95% CI of the average or mean inter-burst duration is 30 092 ms with the average or mean inter-burst duration equal to 25 837 ms. Experimental data generated for fluoxetine, sertraline, JB-1 and squalamine shows that the lower 95% CI of the average or mean inter-burst duration is 21 548 ms and the upper 95% CI of the average or mean inter-burst duration is 29 350 ms with the average or mean inter-burst duration equal to 25 449 ms. The corresponding lower and upper 95% CI values for the individual antidepressant agents are 11 524 and 44 175 ms for fluoxetine, 16 160 and 36 270 ms for sertraline, 13 108 and 35 688 ms for JB-1 and 11 233 and 34 229 ms for squalamine.

The average inter-pulse duration of the vagus nerve code should be within an interval of 180 and 1 600 ms. In a preferred embodiment, the controller 16, 26 is configured to control the pulse generator 14, 24 to generate the bursts of vagus nerve stimulation pulses having the average inter-pulse duration selected within an interval of from 380 to 990 ms, more preferably within an interval of from 490 to 650 ms.

Experimental data generated for the three antidepressant agents fluoxetine, sertraline and JB-1 shows that the lower 95% CI of the average or mean inter-pulse duration is 499 ms and the upper 95% CI of the average or mean inter-pulse duration is 648 ms with the average or mean inter-pulse duration equal to 574 ms. Experimental data generated for fluoxetine, sertraline, JB-1 and squalamine shows that the lower 95% CI of the average or mean inter-pulse duration is 498 ms and the upper 95% CI of the average or mean inter-pulse duration is 633 ms with the average or mean inter-pulse duration equal to 565 ms. The corresponding lower and upper 95% CI values for the individual antidepressant agents are 612 and 983 ms for fluoxetine, 386 and 614 ms for sertraline, 431 and 688 ms for JB-1 and 334 and 675 ms for squalamine.

In an embodiment, the vagus nerve code is defined not only by the average inter-burst duration and the average inter-pulse duration but also by an average burst duration. In such an embodiment, the controller 16, 26 is configured to control the pulse generator 14, 24 to generate the bursts of vagus nerve stimulation pulses having an average burst duration selected within an interval of from 240 to 1 630 ms.

In a preferred embodiment, the controller 16, 26 is configured to control the pulse generator 14, 24 to generate the bursts of vagus nerve stimulation pulses having an average burst duration selected within an interval of from 510 to 780 ms, more preferably within an interval of from 560 to 700 ms.

Experimental data generated for the three antidepressant agents fluoxetine, sertraline and JB-1 shows that the lower 95% CI of the average or mean burst duration is 565 ms and the upper 95% CI of the average or mean burst duration is 698 ms with the average or mean burst duration equal to 631 ms. Experimental data generated for fluoxetine, sertraline, JB-1 and squalamine shows that the lower 95% CI of the average or mean burst duration is 566 ms and the upper 95% CI of the average or mean burst duration is 796 ms with the average or mean burst duration equal to 681 ms. The corresponding lower and upper 95% CI values for the individual antidepressant agents are 510 and 771 ms for fluoxetine, 516 and 676 ms for sertraline, 582 and 762 ms for JB-1 and 110 and 1 954 ms for squalamine.

In an alternative, or additional, embodiment, the vagus nerve code is defined not only by the average inter-burst duration and the average inter-pulse duration and optionally by the average burst duration but also by an average inter-pulse duration within the bursts (averageinter-pulse interval within burst or average intra-burst interval). In such an embodiment, the controller 16, 26 is configured to control the pulse generator 14, 24 to generate the bursts of vagus nerve stimulation pulses having an average inter-pulse duration within the bursts selected within an interval of from 70 to 340 ms.

In a preferred embodiment, the controller 16, 26 is configured to control the pulse generator 14, 24 to generate the bursts of vagus nerve stimulation pulses having an average inter-pulse duration within the bursts selected within an interval of from 80 to 140 ms, more preferably within an interval of from 100 to 125 ms.

Experimental data generated for the three antidepressant agents fluoxetine, sertraline and JB-1 shows that the lower 95% CI of the average or mean inter-pulse duration within the bursts is 101 ms and the upper 95% CI of the average or mean inter-pulse duration within the bursts is 121 ms with the average or mean inter-pulse duration within the bursts equal to 111 ms. Experimental data generated for fluoxetine, sertraline, JB-1 and squalamine shows that the lower 95% CI of the average or mean inter-pulse duration within the bursts is 103 ms and the upper 95% CI of the average or mean inter-pulse duration within the bursts is 121 ms with the average or mean inter-pulse duration within the bursts equal to 112 ms. The corresponding lower and upper 95% CI values for the individual antidepressant agents are 101 and 138 ms for fluoxetine, 87 and 110 ms for sertraline, 110 and 136 ms for JB-1 and 98 and 136 ms for squalamine.

In an embodiment, the controller 16, 26 is configured to control the pulse generator 14, 24 to generate the bursts of vagus nerve stimulation pulses having the above-defined average inter-burst duration and the above-defined average inter-pulse duration.

In another embodiment, the controller 16, 26 is configured to control the pulse generator 14, 24 to generate the bursts of vagus nerve stimulation pulses having the above-defined average inter-burst duration, the above-defined average inter-pulse duration and the above-defined average burst duration.

In a further embodiment, the controller 16, 26 is configured to control the pulse generator 14, 24 to generate the bursts of vagus nerve stimulation pulses having the above-defined average inter-burst duration, the above-defined average inter-pulse duration and the above-defined average inter-pulse duration within the burst.

In yet another embodiment, the controller 16, 26 is configured to control the pulse generator 14, 24 to generate the bursts of vagus nerve stimulation pulses having the above-defined average inter-burst duration, the above-defined average inter-pulse duration, the above-defined average burst duration and the above-defined average inter-pulse duration within the burst.

In a particular embodiment, the controller 16, 26 is configured to control the pulse generator 14, 24 to generate the bursts of vagus nerve stimulation pulses having the average inter-burst duration selected within an interval of from 21 500 to 30 100 ms, the average inter-pulse duration selected within an interval of from 490 to 650 ms, the average burst duration selected within an interval of from 560 to 700 ms and the average inter-pulse duration within the bursts selected within an interval of from 100 to 125 ms.

In another particular embodiment, the controller 16, 26 is configured to control the pulse generator 14, 24 to generate the bursts of vagus nerve stimulation pulses having the average inter-burst duration selected within an interval of from 11 500 to 44 200 ms, the average inter-pulse duration selected within an interval of from 610 to 990 ms, the average burst duration selected within an interval of from 510 to 780 ms and the average inter-pulse duration within the bursts selected within an interval of from 100 to 140 ms.

In a further particular embodiment, the controller 16, 26 is configured to control the pulse generator 14, 24 to generate the bursts of vagus nerve stimulation pulses having the average inter-burst duration selected within an interval of from 16 100 to 36 300 ms, the average inter-pulse duration selected within an interval of from 380 to 620 ms, the average burst duration selected within an interval of from 510 to 680 ms and the average inter-pulse duration within the bursts selected within an interval of from 80 to 110 ms.

In yet another particular embodiment, the controller 16, 26 is configured to control the pulse generator 14, 24 to generate the bursts of vagus nerve stimulation pulses having the average inter-burst duration selected within an interval of from 13 100 to 35 700 ms, the average inter-pulse duration selected within an interval of from 430 to 690 ms, the average burst duration selected within an interval of from 580 to 770 ms and the average inter-pulse duration within the bursts selected within an interval of from 110 to 140 ms.

In a further particular embodiment, the controller 16, 26 is configured to control the pulse generator 14, 24 to generate the bursts of vagus nerve stimulation pulses having the average inter-burst duration selected within an interval of from 11 200 to 34 300 ms, the average inter-pulse duration selected within an interval of from 330 to 680 ms, the average burst duration selected within an interval of from 110 to 1 960 ms and the average inter-pulse duration within the bursts selected within an interval of from 90 to 140 ms.

The above presented values of the parameter or variables of the vagus nerve code have been determined based on experiments conducted in mice. Generally, such rodent-based experiments involving vagal nerve stimulation have had good predictive value when later tested in humans. For instance, preclinical research using vagal nerve stimulation in rodent models has been a successful predictor of positive outcomes in clinical trials for, among others, Crohn's disease, metabolic syndrome and rheumatoid arthritis (Pavlov & Tracey, Nat Neurosci 20: 156-166, 2017; Koopman et al., Proc Natl Acad Sci USA 113: 8284-8289, 2016). The predictive effect of mouse models has also been noted in other non-immune neurally-dependent reflexes such as propulsive peristalsis (Keating et al., Toxicol Appl Pharmacol 245: 299-309, 2010).

In an embodiment, the controller 16, 26 is configured to control the pulse generator 14, 24 to generate the bursts of vagus nerve stimulation pulses having the average inter-burst duration selected within an interval of X₁±αX₁, the average inter-pulse duration selected within an interval of X₂±αX₂, optionally the average burst duration selected within an interval of X₃±αX₃, and optionally the average inter-pulse duration within the bursts selected within an interval of X₄±αX₄. In this embodiment, X₁₋₄ denotes the above mentioned average or mean values for the four parameters or variables, i.e., X₁=25 837 ms or 25 449 ms, X₂=574 ms or 565 ms, X₃=631 ms or 681 ms and X₄=111 ms or 112 ms. The parameter α is 0<α<1, preferably 0<α<0.75, such as 0<α<0.7, 0<α<0.65, 0<α<0.6, 0<α<0.55, more preferably 0<α<0.5, 0<α<0.45, 0<α<0.4, 0<α<0.35, 0<α<0.3, 0<α<0.25, 0<α<0.20, 0<α<0.15, 0<α<0.1 or 0<α<0.05.

The duration of each individual pulse would be approximately 1 ms with an amplitude of about 1 to 4 mA as illustrative, but non-limiting, examples. These values together with the duration of the train of bursts per application instance can be determined to achieve a sufficient effectiveness and comfort to the subject.

The device 10, 20 preferably comprises an electrode connector 12, 22 connected to the pulse generator 14, 24 as indicated in FIGS. 1 and 2. This electrode connector 12, 22 is also connectable to at least one stimulation electrode 2.

The at least one stimulation electrode 2 is then configured to be position on or in the subject body in contact with or in connection with the vagus nerve in order to apply the vagus nerve stimulation pulses generated by the pulse generator 14, 24 to the vagus nerve.

In a typical implementation, the electrode connector 12, 22 is connectable to multiple stimulation electrodes 2, such as two stimulation electrodes 2.

In an embodiment, the stimulation electrodes 2 constitute different parts of the housing or case of the device 10, 20. In another embodiment, one of the stimulation electrodes 2 constitutes at least a part of the housing or case of the device 10, 20, whereas another stimulation electrode 2 is provided with a distance from the device 10, 20, such as on a lead or catheter 1 as shown in FIGS. 1 and 2. In a further embodiment, all the stimulation electrodes 2 are provided with a distance from the device 10, 20, such as on the lead or catheter 1, or on separate leads or catheters 1.

In an embodiment, the device 10 is designed to be arranged outside of the subject body, i.e., not implanted. Hence, the device 10 is an external device 10 for vagus nerve stimulation. For instance, the device 10 could be an aural device 10 having stimulation electrodes 2 placed on the skin of the subject in connection with an ear. Such an external device 10 is then capable of electrically stimulating the vagus nerve noninvasively, such as transcutaneously. The external device 10 could, for instance, be provided in a collar designed to be placed around the neck of the subject as disclosed in US 2013/0245486.

In another embodiment, the device 20 is in the form of an implant, i.e., designed to be implanted into the subject body. For instance, the device 20 could be implanted under the skin below the subject's clavicle. A lead 1 from the device 20 is tunneled up to the subject's neck to provide the stimulation electrodes 2 in contact with or in connection with the vagus nerve, typically left vagus nerve at the carotid sheath.

In the latter case, i.e., an implanted device 20, the device 20 preferably comprises a power source for the pulse generator 24 and the controller 26. This power source is typically in the form of a battery 28 as shown in FIG. 2. The external device 10 may also contain a battery as an internal power source although not shown in FIG. 1. However, it is also possible to have an external power source, such as connecting the external device 10 to the external power source using a power cord.

The device 10, 20 for vagus nerve stimulation could be any known vagus nerve stimulator, either implantable or provided externally to the subject body, but which includes a pulse generator 14, 24 and controller 16, 26 according to the present invention, i.e., capable of producing and applying bursts of vagus nerve stimulation pulses according to the disclosed vagus nerve code.

The device 10, 20 for vagus nerve stimulation can be used to treat various mood disorders in a subject. The subject is preferably a human subject. However, the present invention can also be used for veterinary purpose to treat various mood disorders in other mammals, such as dogs, cats, mice, rats, rabbits, guinea pigs, horses, cows, sheep, goats, primates and monkeys.

In these therapeutic applications, the device 10, 20 generates and applies vagus nerve stimulation that mimics the vagus nerve firing pattern induced in patients subject to successful medication against the mood disorder, such as in connection with administration of antidepressants. Thus, the device 10, 20 is capable of providing vagus nerve stimulation similar to the vagus nerve firing pattern seen in patients treated for the mood disorder using medicaments.

Another aspect of the embodiments relates to a method for treating a mood disorder in a subject, see FIG. 4. The method comprises applying, in step S1 and to the subject, bursts of vagus nerve stimulation pulses having an average inter-burst duration selected within an interval of from 6 300 to 49 000 ms and an average inter-pulse duration selected within an interval of from 180 to 1 600 ms.

The method may optionally involve applying the bursts of vagus nerve stimulation pulses according to any of the above described embodiments for the preferred intervals of the average inter-burst duration and the average inter-pulse duration and optionally also the average burst duration and/or average inter-pulse duration within the bursts.

The method preferably involves using a device 10, 20 as described above and illustrated in FIG. 1 or 2 to apply the bursts of vagus nerve stimulation pulses to the subject.

As mentioned in the foregoing, the vagus nerve code used by the device 10, 20 to apply vagus nerve stimulation pulses or applied to the subject in step S1 to treat a mood disorder mimics the vagus nerve pulses induced by the administration of medicaments against the mood disorder, such as following administration of antidepressants.

This means that vagus nerve code of the embodiments can be used in screening methods in order to identify test agents that may be suitable in treating mood disorders.

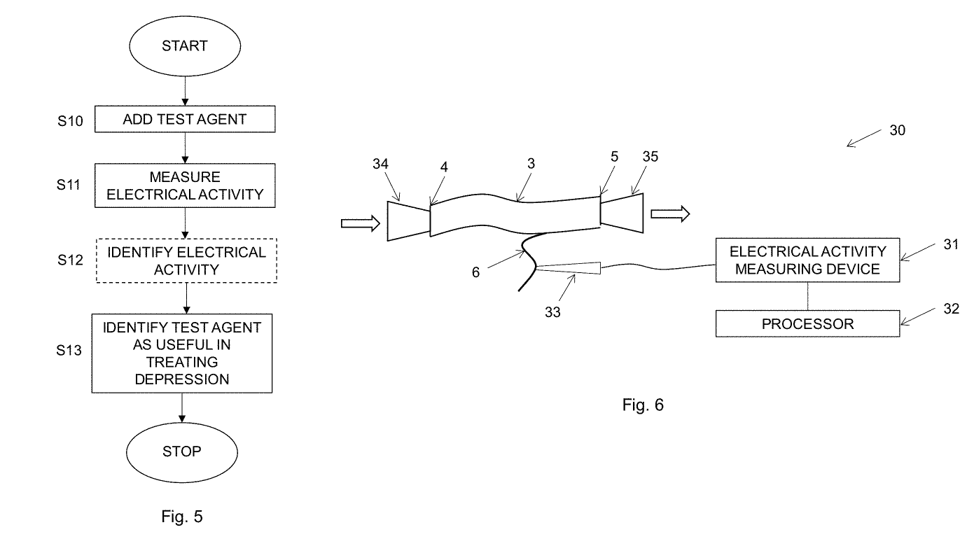

An embodiment therefore relates to an ex vivo screening method, see FIGS. 5 and 6. The method comprises adding, in step S10, a test agent to the lumen of a jejunal segment 3 comprising attached mesenteric nerve tissue 6. The method also comprises measuring, in step S11, electrical activity of the mesenteric nerve tissue 6. The method further comprises identifying, in step S13, the test agent as useful in treating a mood disorder in a subject if the measured electrical activity comprises bursts of nerve pulses having an average inter-burst duration within an interval of from 6 300 to 49 000 ms and an average inter-pulse duration within an interval of from 180 to 1 600 ms.

In a particular embodiment, the identification in step S13 is performed by determining whether the measured electrical activity has parameters or variables according to any of the previously described embodiments of the vagus nerve code, i.e., having the average inter-burst duration and the average inter-pulse duration and optionally also the average burst duration and/or average inter-pulse duration within the bursts within any of the above described preferred intervals.

In an embodiment, the method comprises an additional, optional step S12 as shown in FIG. 5. This step S12 comprises identifying or determining electrical activity from at least one individual single vagal nerve fiber based on the measured electrical activity. In this embodiment, step S13 comprises identifying the test agent as useful in treating the mood disorder in the subject if the electrical activity from the at least one individual single vagal nerve fiber comprises bursts of nerve pulses having the average inter-burst duration within the interval of from 6 300 to 49 000 ms and the average inter-pulse duration within the interval of from 180 to 1 600 ms.

In this embodiment, the electrical activity from single vagal nerve fibers is discriminated and identified to thereby obtain individual waveforms or trains of bursts of vagal single nerve fibers rather than from a whole population of vagal nerve fibers. The electrical activity identified in step S12 thereby represents single vagal nerve fiber responses to the added test agent.

The addition of the test agent in step S10 is preferably performed by providing a luminal perfusion comprising the test agent between respective ends 4, 5 of the jejunal segment 3. In such a case, the ends 4, 5 of the jejunal segment 3 are preferably cannulated with plastic tubing 34, 35 as indicated in FIG. 6.

The electrical activity of the mesenteric nerve tissue 6 is, in an embodiment, measured using a pipette 33 acting as a patch-clamp electrode.

In an embodiment, step S13 also comprises identifying the test agent as not useful in treating the mood disorder in the subject if the measured electrical activity comprises bursts of nerve pulses having the average inter-burst duration outside of the interval of from 6 300 to 49 000 ms and/or the average inter-pulse duration outside of the interval of from 180 to 1 600 ms. Thus, the method can also be used to identify test agents that are not likely to be effective in treating mood disorders since they result in an electrical activity different from the vagus nerve code of the embodiments.

A particular aspect of the embodiments relates to an ex vivo method. The method comprises steps S10 to S12 as shown in FIG. 5. The method comprises adding, in step S10, adding a test agent to the lumen of a jejunal segment comprising attached mesenteric nerve tissue. The method also comprises measuring electrical activity of the mesenteric nerve tissue in step S11. The method further comprises identifying or determining, in step S12, electrical activity from at least one individual single vagal nerve fiber based on the measured electrical activity. This step S12 thereby preferably comprises processing the measured electrical activity in order to get electrical activity from individual single vagal nerve fibers rather than from a whole population of vagal nerve fibers.

A related aspect of the embodiments defines an ex vivo screening system 30. The system 30 comprises a jejunal segment 3 comprising attached mesenteric nerve tissue 6 and having a first end 4 and a second end 5. The system 30 also comprises a first tubing 34 attached to the first end 4 of the jejunal segment and configured to receive a test agent. The system 30 further comprises a second tubing 35 attached to the second end 5 of the jejunal segment 3 and configured to output the test agent, i.e., after having passed through the lumen of the jejunal segment 3. The system 30 additionally comprises an electrical activity measuring unit 31 configured to measure electrical activity of the mesenteric nerve tissue 6. A processor 32 of the system 30 is configured to determine an average inter-burst duration and an average inter-pulse duration of bursts of nerve pulses from the measured electrical activity. The processor 32 is also configured to identify the test agent as useful in treating a mood disorder in a subject if the average inter-burst duration is within an interval of from 6 300 to 49 000 ms and the average inter-pulse duration is within an interval of from 180 to 1 600 ms.

In an embodiment, the processor 32 is also configured to determine an average burst duration and/or an average inter-pulse duration within the bursts. The processor 32 may be configured to identify the test agent as useful in treating the mood disorder if the average parameter or variable values are within any of the disclosed embodiments for the preferred intervals of the average inter-burst duration and the average inter-pulse duration and optionally also the average burst duration and/or average inter-pulse duration within the bursts.

In an embodiment, the processor 32 is configured to identify or determine electrical activity from at least one individual single vagal nerve fiber based on the measured electrical activity. In this embodiment, the processor 32 is also configured to identify the test agent as useful in treating the mood disorder in the subject if the electrical activity from the at least one individual single vagal nerve fiber comprises bursts of nerve pulses having the average inter-burst duration within the interval of from 6 300 to 49 000 ms and the average inter-pulse duration within the interval of from 180 to 1 600 ms.

In an embodiment, the processor 32 is configured to identify the test agent as not useful in treating the mood disorder in the subject if the measured electrical activity comprises bursts of nerve pulses having the average inter-burst duration outside of the interval of from 6 300 to 49 000 ms and/or the average inter-pulse duration outside of the interval of from 180 to 1 600 ms.

Another embodiments defines an ex vivo system 30. The system 30 comprises a jejunal segment 3 comprising attached mesenteric nerve tissue 6 and having a first end 4 and a second end 5. The system 30 also comprises a first tubing 34 attached to the first end 4 of the jejunal segment and configured to receive a test agent. The system 30 further comprises a second tubing 35 attached to the second end 5 of the jejunal segment 3 and configured to output the test agent, i.e., after having passed through the lumen of the jejunal segment 3. The system 30 additionally comprises an electrical activity measuring unit 31 configured to measure electrical activity of the mesenteric nerve tissue 6. A processor 32 of the system 30 is configured to determine or identify electrical activity from at least one individual single vagal nerve fiber based on the measured electrical activity. The processor 32 is also configured to determine an average inter-burst duration and an average inter-pulse duration of bursts of nerve pulses from the determined or identified electrical activity.

The above described screening method and system use ex vivo tissue in the form of a jejunal segment from a test animal to screen for test agents suitable in treating mood disorder by verifying whether the test agents are capable of inducing nerve pulses according to the vagus nerve code of the embodiments.

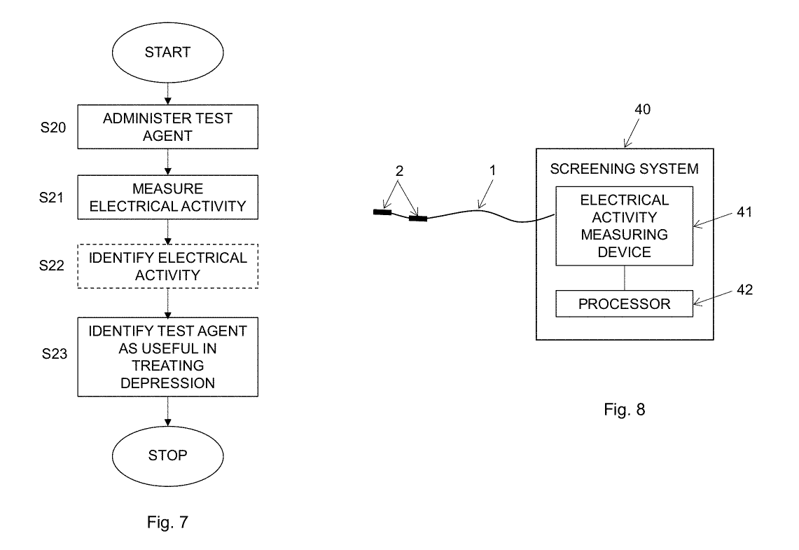

The screening may alternatively be performed in vivo in a test subject. FIG. 7 illustrates such an in vivo screening method. The method comprises administering, in step S20, a test agent to a test subject. Electrical activity of the vagus nerve of the test subject is measured in step S21. The method also comprises identifying, in step S23, the test agent as useful in treating a mood disorder in a subject if the measured electrical activity comprises bursts of vagus nerve pulses having an average inter-burst duration within an interval of from 6 300 to 49 000 ms and an average inter-pulse duration within an interval of from 180 to 1 600 ms.

In a particular embodiment, the identification in step S23 is performed by determining whether the measured electrical activity has parameters or variables according to any of the previously described embodiments of the vagus nerve code, i.e., having the average inter-burst duration and the average inter-pulse duration and optionally also the average burst duration and/or average inter-pulse duration within the bursts within any of the above described preferred intervals.

In an embodiment, the method comprises an additional, optional step S22 as shown in FIG. 7. This step S22 comprises identifying or determining electrical activity from at least one individual single vagal nerve fiber based on the measured electrical activity. In this embodiment, step S23 comprises identifying the test agent as useful in treating the mood disorder in the subject if the electrical activity from the at least one individual single vagal nerve fiber comprises bursts of nerve pulses having the average inter-burst duration within the interval of from 6 300 to 49 000 ms and the average inter-pulse duration within the interval of from 180 to 1 600 ms.

In an embodiment, step S23 also comprises identifying the test agent as not useful in treating the mood disorder in the subject if the measured electrical activity comprises bursts of nerve pulses having the average inter-burst duration outside of the interval of from 6 300 to 49 000 ms and/or the average inter-pulse duration outside of the interval of from 180 to 1 600 ms.

A particular aspect of the embodiments relates to an in vivo method. The method comprises steps S20 to S22 as shown in FIG. 7. The method comprises administering a test agent to a test subject in step S20. The method also comprises measuring electrical activity of the vagus nerve of the test subject in step S21. The method further comprises identifying electrical activity from at least one individual single vagal nerve fiber based on the measured electrical activity in step S22. This step S22 thereby preferably comprises processing the measured electrical activity in order to get electrical activity from individual single vagal nerve fibers rather than from a whole population of vagal nerve fibers.

A related aspect of the embodiments defines a screening system 40, see FIG. 8. The screening system 40 comprises an electrical activity measuring device 41 configured to measure electrical activity of the vagus nerve of a test subject, to which a test agent has been administered. The screening system 40 also comprises a processor 42 configured to determine an average inter-burst duration and an average inter-pulse duration of bursts of vagus nerve pulses from the measured electrical activity. The processor 42 is also configured to identify the test agent as useful in treating a mood disorder in a subject if the average inter-burst duration is within an interval of from 6 300 to 49 000 ms and the average inter-pulse duration is within an interval of from 180 to 1 600 ms.

In an embodiment, the processor 42 is also configured to determine an average burst duration and/or an average inter-pulse duration within the bursts. The processor 42 may be configured to identify the test agent as useful in treating the mood disorder if the average parameter or variable values is within any of the disclosed embodiments for the preferred intervals of the average inter-burst duration and the average inter-pulse duration and optionally also the average burst duration and/or average inter-pulse duration within the bursts.

In an embodiment, the processor 42 is configured to identify or determine electrical activity from at least one individual single vagal nerve fiber based on the measured electrical activity. In this embodiment, the processor 42 is also configured to identify the test agent as useful in treating the mood disorder in the subject if the electrical activity from the at least one individual single vagal nerve fiber comprises bursts of nerve pulses having the average inter-burst duration within the interval of from 6 300 to 49 000 ms and the average inter-pulse duration within the interval of from 180 to 1 600 ms.

In an embodiment, the processor 42 is configured to identify the test agent as not useful in treating the mood disorder in the subject if the measured electrical activity comprises bursts of nerve pulses having the average inter-burst duration outside of the interval of from 6 300 to 49 000 ms and/or the average inter-pulse duration outside of the interval of from 180 to 1 600 ms.

An embodiment relates to a screening system 40. The screening system 40 comprises an electrical activity measuring device 41 configured to measure electrical activity of the vagus nerve of a test subject, to which a test agent has been administered. The screening system 40 also comprises a processor 42 configured to identify or determine electrical activity from at least one individual single vagal nerve fiber based on the measured electrical activity. The processor 42 is also configured to determine an average inter-burst duration and an average inter-pulse duration of bursts of vagus nerve pulses from the identified or determined electrical activity.

The screening system 40 could be in the form of an implanted system or device or an external, i.e., non-implanted, system or device as long as the electrical activity measuring device 41 is capable of measuring the electrical activity of the vagus nerve. In the latter case, the electrical activity measuring device 41 measures the electrical activity through the skin, i.e., so-called transcutaneous electrical measurement.

The electrical activity measuring device 41 is preferably connectable to at least one electrode 2 used to capture the electrical activity of the vagus nerve. This at least one electrode 2 could be provided as a part of the case or housing and/or provided at a distance from the case or housing, such as on a lead or catheter 1 as described in the foregoing in connection with FIGS. 1 and 2.

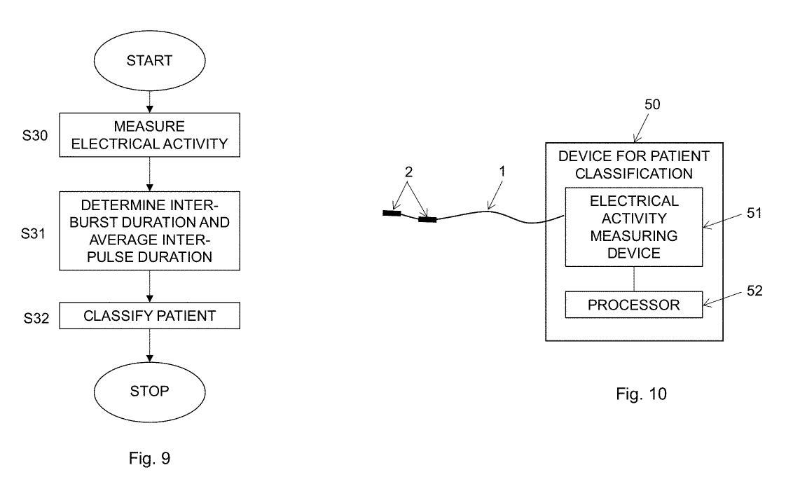

Yet another aspect of the embodiments relates to a method for patient classification, see FIG. 9. The method comprises measuring, in step S30, electrical activity of the vagus nerve of a patient. An average inter-burst duration and an average inter-pulse duration of bursts of vagus nerve pulses are determined in step S32 from the measured electrical activity. The method also comprises classifying, in step S33, the patient as tentative suffering from a mood disorder if the average inter-burst duration is outside of an interval of from 6 300 to 49 000 ms and the average inter-pulse duration selected is outside of an interval of from 180 to 1 600 ms.

In particular embodiment, the step S32 also comprises determining an average burst duration and/or an average inter-pulse duration within the bursts from the measured electrical activity. The classification in step S33 may be performed by determining whether the at least two parameters or variables determined in step S32 is outside of any the previously described preferred intervals for the vagus nerve code, i.e., having the average inter-burst duration and the average inter-pulse duration and optionally also the average burst duration and/or average inter-pulse duration within the bursts outside any of the above described preferred intervals.

In an embodiment, the method comprises an additional, optional step S31 as shown in FIG. 9. This step S31 comprises identifying electrical activity from at least one individual single vagal nerve fiber based on the measured electrical activity. In this embodiment, step S32 comprises determining the average burst duration and/or the average inter-pulse duration within the burst based on the electrical activity from the at least one individual single vagal nerve fiber.

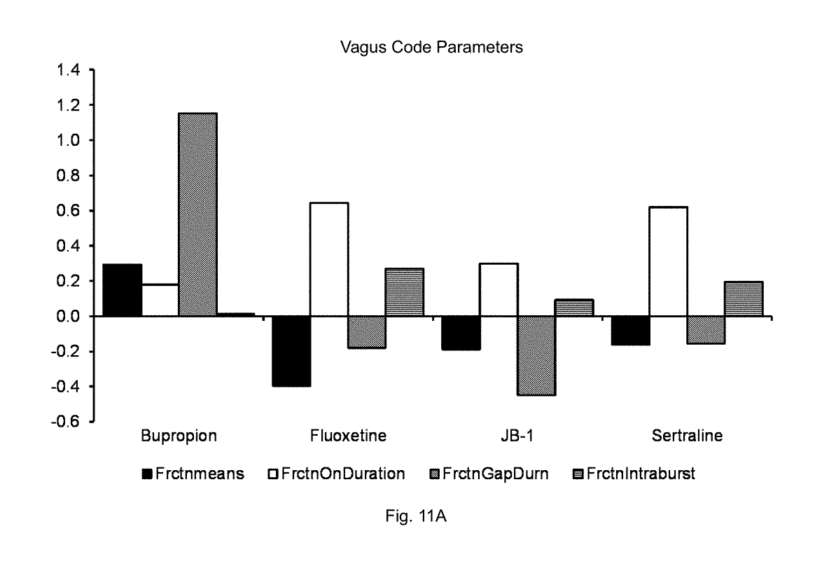

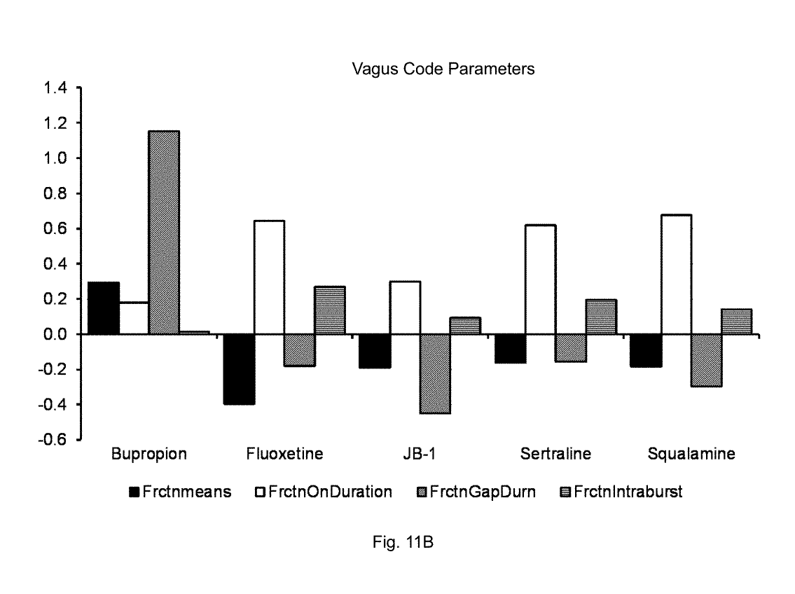

A related aspect defines a device 50 for patient classification, see FIG. 10. The device 50 comprises an electrical activity measuring device 51 configured to measure electrical activity of the vagus nerve of a patient. The device 50 also comprises a processor 52 configured to determine an average inter-burst duration and an average inter-pulse duration of bursts of vagus nerve pulses from the measured electrical activity. The processor 52 is also configured to classify the patient as tentative suffering from a mood disorder if the inter-burst duration is outside of an interval of from 6 300 to 49 000 ms and the average inter-pulse duration selected is outside of an interval of from 180 to 1 600 ms.

In particular embodiment, the processor 52 is also configured to determine an average burst duration and/or an average inter-pulse duration within the bursts from the measured electrical activity. The processor 52 may perform the classifying by determining whether the at least two parameters or variables is outside of any the previously described preferred intervals for the vagus nerve code, i.e., having the average inter-burst duration and the average inter-pulse duration and optionally also the average burst duration and/or average inter-pulse duration within the bursts outside any of the above described preferred intervals.

In an embodiment, the processor 52 is configured to identify electrical activity from at least one individual single vagal nerve fiber based on the measured electrical activity. In this embodiment, the processor 52 is configured to determine the average burst duration and/or the average inter-pulse duration within the burst based on the electrical activity from the at least one individual single vagal nerve fiber.

The device 50 could be in the form of an implanted device or an external, i.e., non-implanted, device as long as the electrical activity measuring device 51 is capable of measuring the electrical activity of the vagus nerve. In the latter case, the electrical activity measuring device 51 measures the electrical activity through the skin, i.e., so-called transcutaneous electrical measurement.

The electrical activity measuring device 51 is preferably connectable to at least one electrode 2 used to capture the electrical activity of the vagus nerve. This at least one electrode 2 could be provided as a part of the case or housing and/or provided at a distance from the case or housing, such as on a lead or catheter 1 as described in the foregoing in connection with FIGS. 1 and 2.

The mood disorder mentioned in the foregoing can be any mood disorder that can be treated via vagus nerve stimulation. Non-limiting, but preferred, examples of such mood disorders include a bipolar disorder, such as bipolar I disorder and bipolar II disorder; cyclothymic disorder; dysthymic disorder; seasonal affective disorder; depression or a depressive disorder, such as a major depressive disorder, major depressive episode, minor depressive disorder, atypical depression, melancholic depression, psychotic depression, postpartum depression, and recurrent brief depressive disorder; mood disorders due to a general medical condition; substance-induced mood disorders; panic attacks; anxiety; and obsessive compulsive disorder. In an embodiment, the mood disorder is depression or a depressive disorder, or anxiety.

Non-limiting, but illustrative, examples of major depressive disorders include melancholia, psychotic depression, antenatal and postnatal depression.

Mood disorders can be diagnosed using criteria found in the American Psychiatric Association's revised fourth edition of the Diagnostic and Statistical Manual of Mental Disorders (DSM-IV-TR), and the WHO'S International Statistical Classification of Diseases and Related Health Problems (ICD-10). DSM-IV sets forth diagnostic criteria, descriptions and other information to guide the classification and diagnosis of mental disorders and is commonly used in the field of neuropsychiatry.

Although the present embodiments is in particular suitable for the treatment of mood disorders, the present invention can generally be used to treat any disorder or disease generally treated with selective serotonin reuptake inhibitors (SSRIs). Non-limiting, but illustrative, examples of such disorders or diseases include anxiety disorders, bipolar disorders, body dysmorphic disorders, borderline disorder, bowel disorders, depression, discontinuation syndrome, eating disorders, general anxiety disorder, major depressive disorder, obsessive compulsion disorder, panic disorders, personality disorders, post-traumatic stress disorders, premenstrual dysphoric disorder, social anxiety disorders, and social phobia.

An aspect relates to a method for identification of a vagus nerve code. The method comprises steps S10 to S12 as shown in FIG. 5 or steps S20 to S22 as shown in FIG. 7. In this aspect, the test agent added in step S10 or administered in step S20 is a medicament known to be useful in treating, inhibiting and/or preventing a disease or disorder. The method also comprises determining values of multiple vagus code variables or parameters based on the electrical activity from at least one individual single vagal nerve fiber to obtain the vagus nerve code representative of the particular medicament and preferably also representative of the disease or disorder.

Hence, in an embodiment, the method comprises adding, in step S10, a medicament to the lumen of a jejunal segment comprising attached mesenteric nerve tissue. The method also comprises measuring, in step S11, electrical activity of the mesenteric nerve tissue and identifying, in S12, electrical activity from at least one individual single vagal nerve fiber based on the measured electrical activity. In another embodiment, the method comprises administering a medicament to a test subject in step S20. The method also comprises measuring, in step S21, electrical activity of the vagus nerve of the test subject and identifying, in S22, electrical activity from at least one individual single vagal nerve fiber based on the measured electrical activity.

Thus, the method of this aspect is not necessarily related to screening for test agents that are suitable for treating above discussed mood disorders. In clear contrast, different disorders or diseases have respective statistically discernible single vagus nerve fiber firing patterns that are representative for the particular disorder or disease.

In a particular embodiment, a library of such vagus code variables or parameters and associated variable or parameter intervals has been generated and can be used in the screening method. For instance, the library could include a first set of vagus code variables or parameters and associated variable or parameter intervals representative for medicaments useful in treating a first disease or disorder, a second set of vagus code variables or parameters and associated variable or parameter intervals representative for medicaments useful in treating a second disease or disorder, and so on. Once a test agent is available and any medical effect thereof is to be tested, the above described screening method may be conducted to derive values for the multiple vagus code variables or parameters for the test agent. These values may then be compared to the respective defined variable or parameter intervals from the library to see if the test agent produces the same or similar single vagus nerve fiber response as any of the medicaments used to generate the library. If there is match, the test agent is presumed to have the same or similar medical effect as the relevant medicament and could therefore be useful in treating the same disease or disorder as the relevant medicament.

The library can be generated by performing steps S10 to S12 in FIG. 5 or steps S20 to S22 in FIG. 7 using, for a given disease or disorder, one, or preferably, multiple medicaments known to be useful in treating the given disease or disorder. In this method, multiple vagus code variables or parameters are identified by multivariate analysis, preferably multivariate post-hoc analysis, of the electrical activity from the at least one individual single vagal nerve fiber. The method also comprises determining or defining a respective value interval for each of the identified vagus code variables or parameters based on the electrical activity from the at least one individual single vagal nerve fiber.

Generally, the more recordings of electrical activity that are generated and the more medicaments that are tested for a given disease or disorder, the higher accuracy in identifying the vagus code variables or parameters and in determining representative value intervals.

The above described method was conducted on the agent squalamine (3(3β,5α,7α,24R)-3-({3-[(4-aminobutyl)amino]propyl}amino)-7-hydroxycholestan-24-yl hydrogen sulfate). Squalamine is a cationic amphipathic aminosterol first isolated from the dogfish shark in 1993 and has broad-spectrum antibiotic and antiviral properties. Squalamine displaces membrane-bound proteins by neutralizing the negative electrostatic intracellular membrane. In similar fashion, squalamine displaces α-synuclein from binding lipid membranes of vesicles, preventing α-synuclein aggregation, which has led to its Phase 2a clinical trial in 50 patients with Parkinson's disease (PD) and severe constipation. There were positive beneficial effects of squalamine on relief of constipation with an increasing dosage schedule. It was also noted that some neurological symptoms in PD patients like hallucinations, sleep and circadian rhythm disturbances were attenuated or prevented while patients were taking the drug, suggesting the reversal of patients' neurological symptoms. In experimental support of these clinical findings, squalamine, when applied intraluminally in mice, have prokinetic effects on colonic motility and reversed age and loperamide-related dysmotility and squalamine increases vagal afferent firing frequency that had been reduced in aged mice. Since vagal stimulation of a generic nature also appears to have long-term beneficial effect in patients with treatment-resistant depression, the potent stimulation of vagal afferent firing rate by squalamine prompted the analysis of the vagal code for squalamine.

Experimental data as presented herein showed that squalamine resulted in almost identical vagus code as sertraline and very similar to that of fluoxetine and JB-1. Hence, the particular vagal nerve code of vagal afferent firing rate by squalamine indicated that squalamine should be useful as antidepressant.

### EXAMPLES

### Example 1—Ex Vivo Vagal Nerve Fiber Recording

Adult BALB/c mice were killed by cervical dislocation. Then a 2-3 cm long jejunal segment with attached mesenteric tissue was removed and the oral and anal ends of the jejunal segment were cannulated with plastic tubing, gently emptied of contents by perfusing the lumen with Krebs buffer using a 3 mL plastic syringe whose tip was inserted into the oral opening. The cleaned jejunal segment was placed into a polystyrene petri dish (Falcon 351006, 50 mm×9 mm, Corning, N.Y., USA) filled with Krebs buffer of the following composition (in mM): 118 NaCl, 4.8 KCl, 25 NaHCO₃, 1.0 NaH₂PO₄, 1.2 MgSO₄, 11.1 glucose, and 2.5 CaCl₂) bubbled with carbogen (95% 02 and 5% 002). The bottom of the dish was previously lined 1-2 mm deep with cured silicone elastomer SYLGARD®, Dow Corning, MI, USA.

Next the mesenteric nerve bundle was isolated by careful dissection under a stereomicroscope (Leika) using pointed number 5 forceps (Fine Science Tools), and the dish and contents were transferred to an inverted microscope (Nikon).

The gut segment and attached mesenteric tissue was then pinned linearly, without stretching, from oral to anal in the dish by pushing fine insect pins through the adherent mesentery into the silicone elastomer SYLGARD®. The lumen was gravity perfused (1 ml/min) at room temperature (20-25° C.), with oxygenated Krebs buffer and using several Mariotti bottles (McCarthy, Science 80: 100, 1934) attached to a plastic manifold (World Precision Instruments, Sarasota, Fla., USA). The serosal compartment was separately perfused with pre-warmed (35° C.) Krebs buffer at 4 ml/min. 3 μM nicardipine hydrochloride was added to the latter Krebs buffer to paralyze the smooth muscle to isolate chemosensory afferent signals from motility-related vagal responses (Perez-Burgos et al., Am J Physiol Gastrointest Liver Physiol 304: G211-220, 2013).

The nerve bundle was gently sucked onto a glass pipette attached to a patch-clamp electrode holder (CV-7B; Molecular Devices, Sunnyvale, Calif.), and extracellular nerve recordings were performed using a Multi-Clamp 700B amplifier and Digidata 1440A signal converter (Molecular Devices). Electrical signals were bandpass-filtered at 0.1-2 kHz, sampled at 20 kHz, and stored on a personal computer running pClamp 10 software (Molecular Devices) for post hoc analysis. Constitutive multiunit electrical activity was always recorded from the mesenteric nerve bundle even when the lumen was perfused with only Krebs buffer. Baseline recordings with Krebs buffer in the lumen were performed for 15 min, after which the luminal perfusate was switched to one containing Krebs buffer with substances to be tested (fluoxetine 30 μM, sertraline 10 μM, bupropion (WELLBUTRIN®) 10 μM, Lactobacillus rhamnosus JB-1 10⁹ cfu/mL, or squalamine 10 μM). Recording in the presence of test substances was performed for up to 40 min after which the luminal perfusate was again switched to Krebs buffer and recording continued for up to 30 min.

### Example 2—Post Hoc Analysis of Vagus Nerve Fiber Recordings

Single units representing discharge from individual single vagal fibers were discriminated and identified by their unique spike waveform shape and amplitude (Rong et al., J Physiol 560: 867-881, 2004) using Dataview computer software (Heitler, Journal of Undergraduate Neuroscience Education 6: A1-A7, 2007). Dataview uses principal component analysis to sort the recorded multiunit spikes into individual single unit categories according to shape and amplitude (Heitler, Journal of Neuroscience Methods 185: 151-164, 2009).

Single units were then separated according to their shape in Dataview and displayed with unique color codes, each color representing a “channel” in the program for each unit type.

Control and treatment periods were identified from event markers inserted at the time of recording. Then, for each channel (color-coded unit), spike bursts were detected using the “Poisson surprise” method devised by Legéndy & Salcman (Legendy & Salcman, J Neurophysiol 53: 926-939, 1985). A surprise was defined as −log 10(p), where p is the probability of a set of events occurring this close together by chance. Thus, a surprise value of 2, which we used, reflects a p value of 0.01 (highly significant).

For each single unit channel, and for each control and treatment period, the average spike interval (1/average frequency) corresponding to inter-pulse duration, interburst intervals (GapDur) corresponding to inter-burst duration, burst duration (OnDur) corresponding to burst duration, and intraburst intervals corresponding inter-pulse duration within the bursts were measured using event parameter histogram module available in Dataview.

The results of experiments conducted on different mice using a variety of treatments were then pasted in an Excel spreadsheet for further statistical analysis using the Excel add-in Real Statistics Resource Pack software (Release 5.4). The values of each of the four dependent variable were converted into fractional changes for each independent variable (fluoxetine, sertraline, JB-1, bupropion, squalamine) as follows: (treatment (drug in lumen)−control (Krebs buffer in lumen))/control (Krebs in lumen). This gives the fractional change evoked by each drug.

Because there are multiple dependent variables and multiple independent variables, the appropriate statistical test was a multivariate analysis of variance (MANOVA) test. This performed in Real Statistics with the appropriate contrasts set. For example, fluoxetine and sertraline and JB-1 versus bupropion. The Wilk's Lambda test statistic gave the probability that there was a difference for these contrasts, in our case p=2.36E-08 (highly significant, better than 5 sigma), see Table 1. In Table 1, df1 and df2 are the degrees of freedom in the two separate dimension in the data table. F represents the Snedecor's F distribution statistic used to calculate the probability, i.e., p-value. The effect for MANOVA is the partial eta-squared statistic commonly used as an index for the effect size in MANOVA.

We also wanted to know, to which of the independent variables, the calculated categorical difference could be statistically attributed. This was done by calculating the Bonferroni confidence intervals for the independent variables. The Bonferroni confidence interval was equal to the (mean−standard error)/critical t-value for 0.05 significance for the lower bound, and (mean+standard error)/critical t-value for the upper bound (http://www.real-statistics.com/multivariate-statistics/multivariate-analysis-of-variance-manova/manova-follow-up-contrasts/).

TABLE 1
probability parameters

| | |
|---|---|
| No. of dependent variables | 4 |
| No. of independent variables | 4 |
| df1 | 4 |
| df2 | 100 |
| F | 12.62617 |
| p-value | 2.36E−08 |
| effect | 2.158139 |

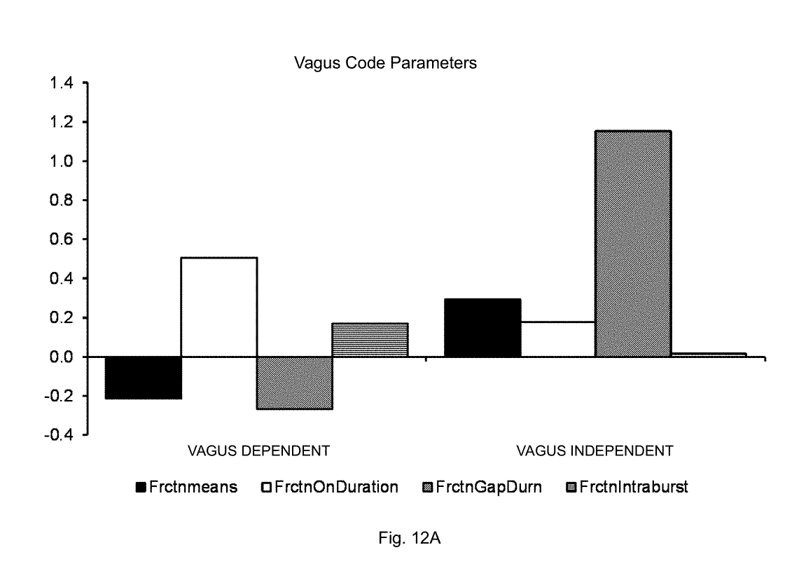

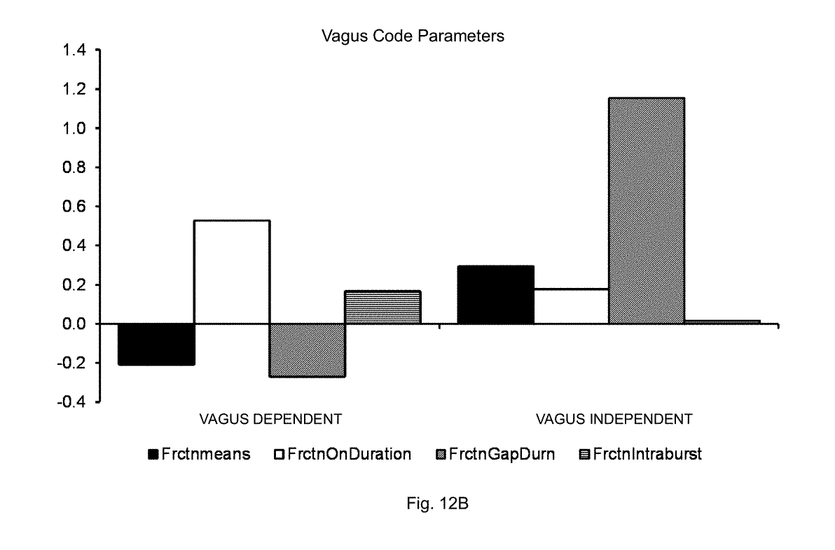

FIG. 11A illustrates the fractional changes of each of the four vagus nerve code variables or parameters for three vagally dependent antidepressants, i.e., fluoxetine, sertraline, JB-1, and for one vagally independent antidepressant, i.e., bupropion, with FIG. 11B showing the fractional changes with squalamine added. FIG. 12A illustrates the corresponding fractional changes for the combined results for fluoxetine, sertraline and JB-1 as vagally dependent antidepressant and for bupropion as vagally independent antidepressant with FIG. 12B showing the results when squalamine is added. These values are further presented in Table 2A (without squalamine) and Table 2B (with squalamine), and the Bonferroni confidence intervals are presented in Table 3A (without squalamine) and Table 2B (with squalamine).

TABLE 2A
fractional changes

| | Fraction | | | Fraction |
|---|---|---|---|---|
| | average | Fraction | Fraction | inter-pulse |
| | inter-pulse | burst | inter-burst | duration |
| | duration | duration | duration | within bursts |
| Fluoxetine | −0.39638989 | 0.642443183 | −0.179524975 | 0.270516033 |
| JB-1 | −0.18811704 | 0.298561622 | −0.450183852 | 0.091571924 |
| Sertraline | −0.1626515 | 0.618354176 | −0.155387698 | 0.193609059 |
| Bupropion | 0.29192997 | 0.177805007 | 1.152059703 | 0.015763254 |
| Vagally | −0.21276 | 0.505811 | −0.26723 | 0.169786 |
| dependent | | | | |
| antidepressants | | | | |
| Vagally | 0.29193 | 0.177805 | 1.15206 | 0.015763 |
| independent | | | | |
| antidepressants | | | | |

TABLE 2BA
fractional changes

| | Fraction | | | Fraction |
|---|---|---|---|---|
| | average | Fraction | Fraction | inter-pulse |
| | inter-pulse | burst | inter-burst | duration |
| | duration | duration | duration | within bursts |
| Fluoxetine | −0.39638989 | 0.642443183 | −0.179524975 | 0.270516033 |
| JB-1 | −0.18811704 | 0.298561622 | −0.450183852 | 0.091571924 |
| Sertraline | −0.1626515 | 0.618354176 | −0.155387698 | 0.193609059 |
| Squalamine | −0.18297 | 0.675176 | −0.29571 | 0.139388 |
| Bupropion | 0.29192997 | 0.177805007 | 1.152059703 | 0.015763254 |
| Vagally | −0.209036 | 0.526981 | −0.270786 | 0.165986 |
| dependent | | | | |
| antidepressants | | | | |
| Vagally | 0.29193 | 0.177805 | 1.15206 | 0.015763 |
| independent | | | | |
| antidepressants | | | | |

TABLE 3A
Bonferroni confidence intervals (without squalamine)

| | Fraction | | | Fraction |
|---|---|---|---|---|
| | average | Fraction | Fraction | inter-pulse |
| | inter-pulse | burst | inter-burst | duration |
| | duration | duration | duration | within bursts |
| Lower | −0.69661 | −0.29449 | −2.1819 | −0.07145 |
| Upper | −0.31277 | 0.950503 | −0.65667 | 0.379497 |

TABLE 3B
Bonferroni confidence intervals (with squalamine)

| | Fraction | | | Fraction |
|---|---|---|---|---|
| | average | Fraction | Fraction | inter-pulse |
| | inter-pulse | burst | inter-burst | duration |
| | duration | duration | duration | within bursts |
| Lower | −0.682157776 | −0.242238572 | −2.13761946 | −0.062090682 |
| Upper | −0.319773864 | 0.940590946 | −0.708072595 | 0.362535937 |

A significant difference between vagally dependent and independent stimuli with respect to measured parameters exists where confidence intervals do not overlap 0. Tables 2A, 2B and 3A, 3B clearly indicate that both the average inter-pulse duration (1/firing rate) and the inter-burst durations account for the difference. The difference between vagally dependent and independent stimulation is, thus, statistically accounted for by both average inter-pulse duration and inter-burst duration.

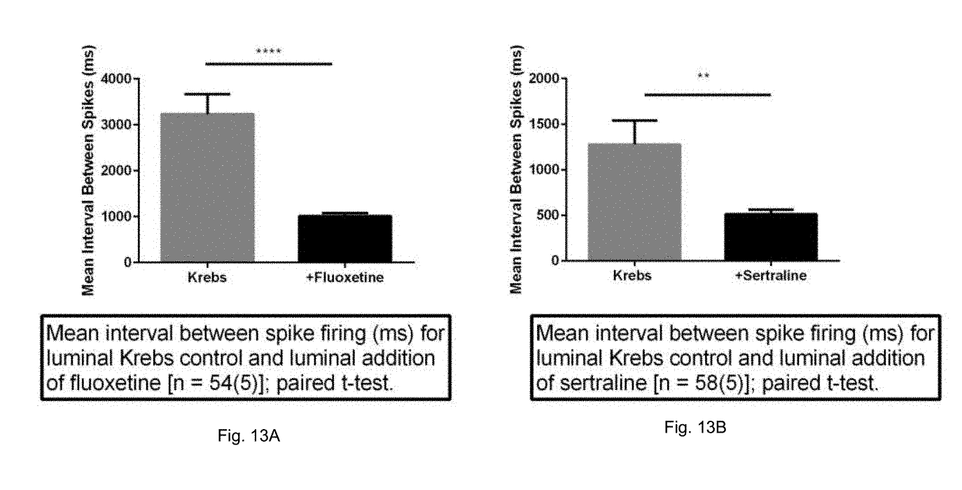

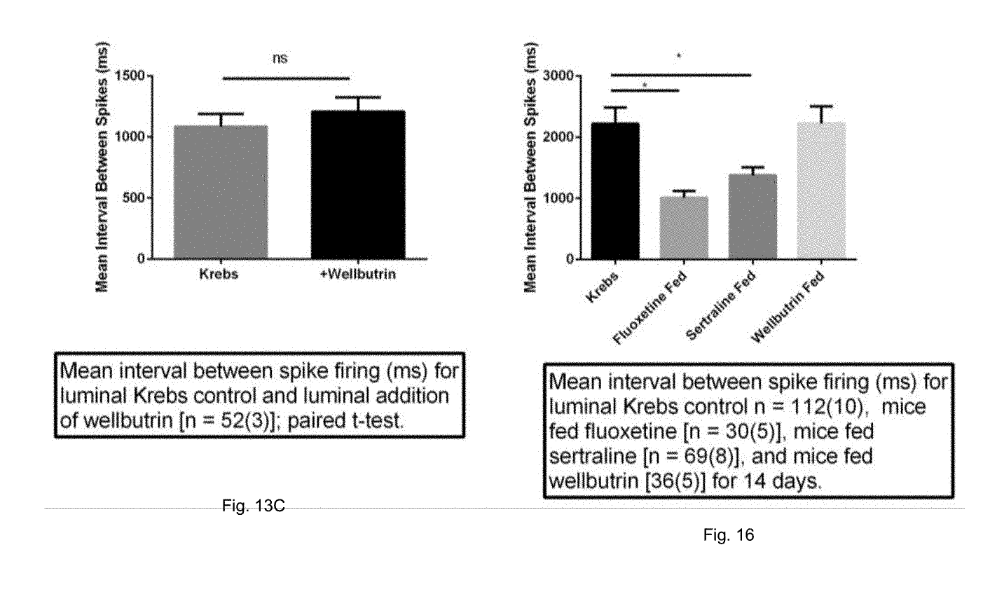

Table 4A and 4B summarize the upper and lower 95% confidence intervals (CIs) for the four antidepressants without squalamine (Table 4A) and for the four antidpepressants with squalamine (Table 4B) and combined 95% CIs for vagally dependent and independent antidepressants in ms. Table 5A and 5B list the corresponding Bonferroni confidence intervals. FIGS. 13A-130 are diagrams illustrating average inter-pulse duration for the two vagally dependent antidepressants fluoxetine and sertraline and for the vagally independent antidepressant bupropion.

TABLE 4A
parameter values

| | | | | | | | Inter-pulse | |
|---|---|---|---|---|---|---|---|---|
| | Average inter- | | | | Inter-burst | | duration | |
| | pulse duration | | Burst duration | | duration | | within bursts | |
| Fluoxetine | 612 | 983 | 510 | 771 | 11524 | 44175 | 101 | 138 |
| JB-1 | 431 | 688 | 582 | 762 | 13108 | 35688 | 110 | 136 |
| Sertraline | 386 | 614 | 516 | 676 | 16160 | 36270 | 87 | 110 |
| Bupropion | 896 | 1081 | 412 | 543 | 49779 | 66105 | 105 | 124 |
| Vagally | 499 | 648 | 565 | 698 | 21582 | 30092 | 101 | 121 |
| dependent | | | | | | | | |
| antidepressants | | | | | | | | |
| Vagally | 896 | 1081 | 412 | 543 | 49779 | 66105 | 105 | 124 |
| independent | | | | | | | | |
| antidepressants | | | | | | | | |

TABLE 4B
parameter values

| | | | | | | | Inter-pulse | |
|---|---|---|---|---|---|---|---|---|
| | Average inter- | | | | Inter-burst | | duration | |
| | pulse duration | | Burst duration | | duration | | within bursts | |
| Fluoxetine | 612 | 983 | 510 | 771 | 11524 | 44175 | 101 | 138 |
| JB-1 | 431 | 688 | 582 | 762 | 13108 | 35688 | 110 | 136 |
| Sertraline | 386 | 614 | 516 | 676 | 16160 | 36270 | 87 | 110 |
| Squalamine | 334 | 675 | 111 | 1954 | 11233 | 34229 | 98 | 136497 |
| Bupropion | 896 | 1081 | 412 | 543 | 49779 | 66105 | 105 | 124 |
| Vagally dependent | 498 | 633 | 566 | 797 | 21548 | 29350 | 103 | 121 |
| antidepressants | | | | | | | | |
| Vagally independent | 896 | 1081 | 412 | 543 | 49779 | 66105 | 105 | 124 |
| antidepressants | | | | | | | | |

TABLE 5A
Bonferroni confidence intervals (without squalamine)

| | Average | | | Inter-pulse |
|---|---|---|---|---|
| | inter-pulse | Burst | Inter-burst | duration |
| | duration | duration | duration | within bursts |
| Lower | −573.502 | 45.42048 | −4563.2 | −19.436 |
| Upper | −255.769 | 262.3524 | −18577.6 | 12.32805 |

TABLE 5B
Bonferroni confidence intervals (with squalamine)

| | Average | | | Inter-pulse |
|---|---|---|---|---|
| | inter-pulse | Burst | Inter-burst | duration |
| | duration | duration | duration | within bursts |
| Lower | −574.575 | 12.768 | −45277 | −17.9429 |
| Upper | −272.012 | 395.182 | −19708 | 12.4115 |

Thus, it was discovered that the antidepressant agents fluoxetine, sertraline and the bacterial strain JB-1 decreased depressive and anxiety behavior in mice and that this effect disappeared when the animals were vagotomized. Decoding the electrical pulse patterns evoked in the vagus nerve when subjected to the above antidepressant agents revealed a specific pulse pattern, a vagus nerve code that signaled the antidepressant effect.

Using post-hoc analysis of hundreds of action potential recordings, four variables were identified that defined a firing pattern induced by the above antidepressant agents. By analyzing these variables, a unique vagal nerve firing pattern was produced which correlated with antidepressant signals.

Multivariate analysis of the results showed that no single variable encoded the observed behavioral effects on anxiety and depression. However, the inter-burst duration and the inter-pulse duration were sufficient to encode the vagus nerve code and provide the antidepressive effects. Including the additional variables burst duration and/or inter-pulse duration within the burst provided an even better definition of the vagus nerve code and the antidepressive effects. In addition, the ratio between specific variables can be also used to define the vagus nerve code.

Because vagal nerve action potentials are “all-or-nothing” events, whether they originate from luminally administered drugs, foodstuffs, or electrical stimulation, the brain will respond in the same way to the same action potential pattern whatever the source. Consequently, the vagus nerve stimulus pattern defined herein would provide an effective antidepressant treatment when programmed into a vagal stimulation device.

Current methods for vagal nerve stimulation typically use stimulation devices that deliver a single frequency ranging from 1 to 30 Hz with a highly stereotyped on-off duty cycle. Natural vagal firing in animals and humans is not stereotyped nor stochastic but consists of patterned bursts that convey information to the brain about the nature of the natural stimulus and have different effects on the brain and mood. Therefore, the vagus nerve code of the present invention, which more closely mimics natural afferent vagal signaling, will allow superior control over mood altering effects evoked by the nerve stimulation. Furthermore, applying vagus nerve stimulation according to the present vagus nerve code will produce fewer side-effects, allow for greater flexibility in fine tuning for specific effects, for example, antianxiety versus antidepression or anti-inflammatory actions and appetitive control.

### Example 3—Tail Suspension Test (TST)

BALB/c mice were orally fed antidepressants fluoxetine (18 mg/kg per day), sertraline (6 mg/kg per day) and bupropion (WELLBUTRIN®) (6 mg/kg per day) in the drinking water for 14 days, or JB-1 (2.5-4×10⁹ bacteria/day) in the drinking water for 28 days.

1 day following oral treatments with the antidepressants, the BALB/c mice were tested for depressive-like behavior with the tail suspension test. Mice were removed from the colony room and moved to a behavioral testing room where they were allowed to habituate for 30 min. Following this period, mice were suspended by the tail using laboratory tape measured to 17 cm (Can et al., Journal of Visualized Experiments 59: e3769, 2012) from a suspension bar in a position whereby they could not escape or hold on to any surfaces. 2 cm of tape was affixed to the mouse tail with the remaining 15 cm used for suspension of the mouse. Animals were left suspended for a period of 6 mins. Animal behavior was video recorded and scored by a blinded observer for freezing behavior. Freezing was calculated as a percentage of total time. Following behavioral testing mice were returned to the housing room and resumed oral treatments for the duration of the study.

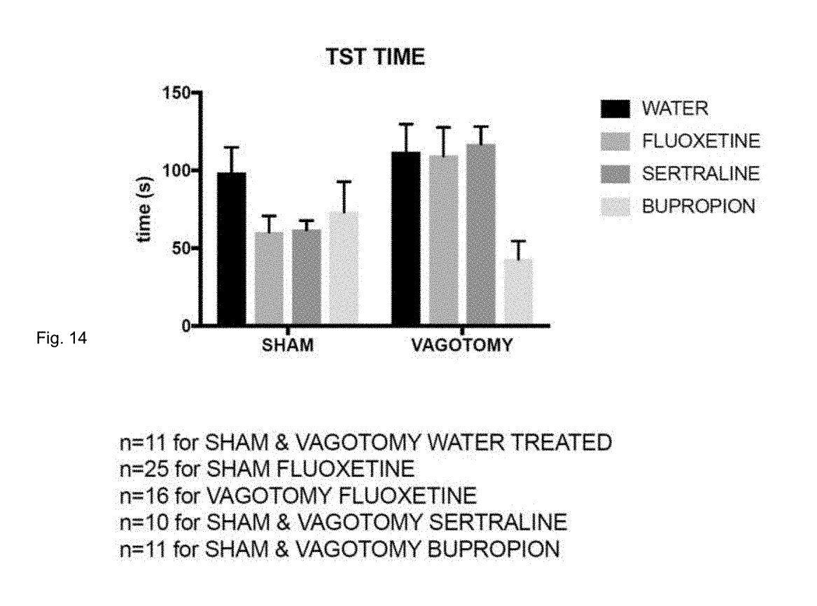

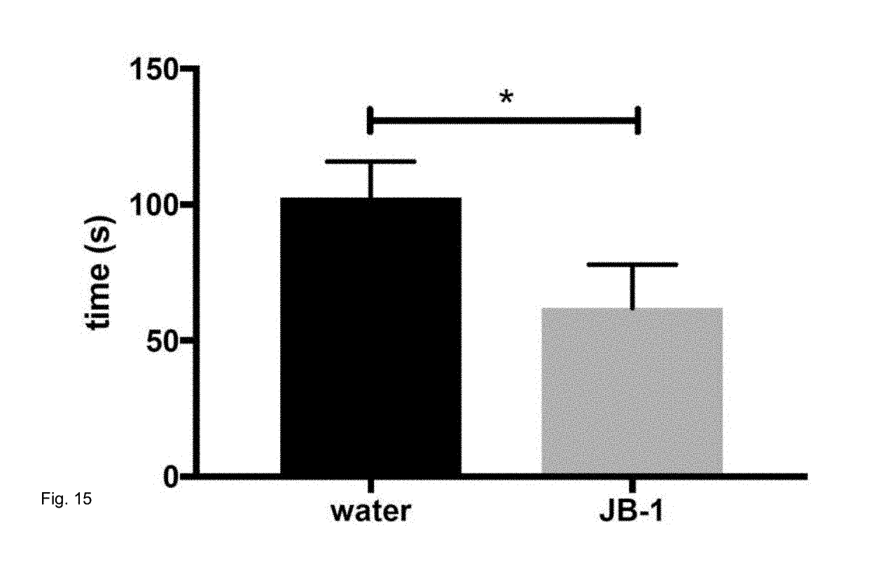

FIG. 14 is a diagram illustrating TST results for the antidepressants in mice and FIG. 15 is a diagram illustrating TST results for JB-1 antidepressant in mice.

The Tail Suspension Test is a simple screening test for the behavioral effects of antianxiety agents and antidepressants in rodents. This test assesses the inter-individual differences in responses to stressful situations measured by duration of immobility. FIG. 14 shows the same dependency of functional action of fluoxetine and sertraline, which are both in extensive clinical use. FIG. 14 clearly shows the effect of fluoxetine and sertraline is dependent on the vagus nerve since vagotomized mice had a significant different average TST time as compared to mice with an intact vagus nerve. Bupropion (WELLBUTRIN®) in clear contrast is shown not to be dependent on the vagus nerve, i.e., its antidepressive effect is present also in vagotmized mice. FIG. 15 shows that there is a significant difference between the TST results for control mice (water) and JB-1 treated mice.

### Example 4—Ex Vivo Vagal Nerve Fiber Recording

The procedure described above in connection with Example 1 and 2 was repeated for the BALB/c mice orally fed with antidepressants fluoxetine (18 mg/kg per day), sertraline (6 mg/kg per day) and bupropion (WELLBUTRIN®) (6 mg/kg per day) in the drinking water for 14 days. Following the oral treatment, the mice were sacrificed and a jejunal segment with mesenteric tissue was recovered as described in Example 1. In this example, the lumens of the jejunal segments were, however, perfused only with KREPS buffer.

FIG. 16 illustrates the mean interval between spike firing, i.e., average inter-pulse durations, for luminal Krebs control, i.e., from control mice that have not been treated with any antidepressants, and for mice fed with fluoxetine, sertraline or bupropion (WELLBUTRIN®). As is clearly seen in the figure, there is a significant difference in average inter-pulse durations between mice treated with fluoxetine or sertraline as compared to control mice but no difference in average inter-pulse durations between mice treated with bupropion as compared to control mice.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A device for vagus nerve stimulation comprising:
a pulse generator configured to generate vagus nerve stimulation pulses; and
a controller configured to control said pulse generator to generate bursts of vagus nerve stimulation pulses having an average inter-burst duration selected within an interval of from 6 300 to 49 000 ms and an average inter-pulse duration selected within an interval of from 180 to 1 600 ms.

2. The device according to claim 1, wherein said controller is configured to control said pulse generator to generate said bursts of vagus nerve stimulation pulses having said average inter-burst duration selected within an interval of from 10 000 to 45 000 ms.

3. The device according to claim 2, wherein said controller is configured to control said pulse generator to generate said bursts of vagus nerve stimulation pulses having said average inter-burst duration selected within an interval of from 21 500 to 30 100 ms.

4. The device according to claim 1, wherein said controller is configured to control said pulse generator to generate said bursts of vagus nerve stimulation pulses having said average inter-pulse duration selected within an interval of from 380 to 990 ms.

5. The device according to claim 4, wherein said controller is configured to control said pulse generator to generate said bursts of vagus nerve stimulation pulses having said average inter-pulse duration selected within an interval of from 490 to 650 ms.

6. The device according to claim 1, wherein said controller is configured to control said pulse generator to generate said bursts of vagus nerve stimulation pulses having an average burst duration selected within an interval of from 240 to 1 630 ms.

7. The device according to claim 6, wherein said controller is configured to control said pulse generator to generate said bursts of vagus nerve stimulation pulses having an average burst duration selected within an interval of from 510 to 780 ms.

8. The device according to claim 7, wherein said controller is configured to control said pulse generator to generate said bursts of vagus nerve stimulation pulses having an average burst duration selected within an interval of from 560 to 700 ms.

9. The device according to claim 1, wherein said controller is configured to control said pulse generator to generate said bursts of vagus nerve stimulation pulses having an average inter-pulse duration within said bursts selected within an interval of from 70 to 340 ms.

10. The device according to claim 9, wherein said controller is configured to control said pulse generator to generate said bursts of vagus nerve stimulation pulses having an average inter-pulse duration within said bursts selected within an interval of from 80 to 140 ms.

11. The device according to claim 10, wherein said controller is configured to control said pulse generator to generate said bursts of vagus nerve stimulation pulses having an average inter-pulse duration within said bursts selected within an interval of from 100 to 125 ms.

12. The device according to claim 1, wherein said controller is configured to control said pulse generator to generate said bursts of vagus nerve stimulation pulses having
said average inter-burst duration selected within an interval of from 21 500 to 30 100 ms;
said average inter-pulse duration selected within an interval of from 490 to 650 ms;
an average burst duration selected within an interval of from 560 to 700 ms; and
an average inter-pulse duration within said bursts selected within an interval of from 100 to 125 ms.

13. The device according to claim 1, further comprising an electrode connector connected to said pulse generator and connectable to at least one stimulation electrode configured to be in contact with or in connection with a vagus nerve of a subject.

14. A method for treating a mood disorder in a subject, said method comprising applying, to said subject, bursts of vagus nerve stimulation pulses having an average inter-burst duration selected within an interval of from 6 300 to 49 000 ms and an average inter-pulse duration selected within an interval of from 180 to 1 600 ms.

15. The method according to claim 14, wherein applying said bursts of vagus nerve stimulation pulses comprises applying, to said subject, bursts of vagus nerve stimulation pulses having
said average inter-burst duration selected within an interval of from 21 500 to 30 100 ms;
said average inter-pulse duration selected within an interval of from 490 to 650 ms;
an average burst duration selected within an interval of from 560 to 700 ms; and
an average inter-pulse duration within said bursts selected within an interval of from 100 to 125 ms.

16. An ex vivo screening system comprising:
a jejunal segment comprising attached mesenteric nerve tissue and having a first end and a second end;
a first tubing attached to said first end of said jejunal segment and configured to receive a test agent;
a second tubing attached to said second end of said jejunal segment and configured to output said test agent;
an electrical activity measuring device configured to measure electrical activity of said mesenteric nerve tissue; and
a processor configured to
determine an average inter-burst duration and an average inter-pulse duration of bursts of nerve pulses from said measured electrical activity; and
identify said test agent as useful in treating a mood disorder in a subject if said average inter-burst duration is within an interval of from 6 300 to 49 000 ms and said average inter-pulse duration is within an interval of from 180 to 1 600 ms.

17. The system according to claim 16, wherein said processor is configured to
identify electrical activity from at least one individual single vagal nerve fiber based on said measured electrical activity; and
identify said test agent as useful in treating said mood disorder in said subject if said electrical activity from said at least one individual single vagal nerve fiber comprises bursts of nerve pulses having said average inter-burst duration within said interval of from 6 300 to 49 000 ms and said average inter-pulse duration within said interval of from 180 to 1 600 ms.

18. A screening system comprising:
an electrical activity measuring device configured to measure electrical activity of the vagus nerve of a test subject, to which a test agent has been administered; and
a processor configured to
determine an average inter-burst duration and an average inter-pulse duration of bursts of vagus nerve pulses from said measured electrical activity; and
identify said test agent as useful in treating a mood disorder in a subject if said average inter-burst duration is within an interval of from 6 300 to 49 000 ms and said average inter-pulse duration is within an interval of from 180 to 1 600 ms.

19. The system according to claim 18, wherein said processor is configured to
identify electrical activity from at least one individual single vagal nerve fiber based on said measured electrical activity; and
identify said test agent as useful in treating said mood disorder in said subject if said electrical activity from said at least one individual single vagal nerve fiber comprises bursts of nerve pulses having said average inter-burst duration within said interval of from 6 300 to 49 000 ms and said average inter-pulse duration within said interval of from 180 to 1 600 ms.
